(19) United States
(12) Patent Application Publication
(10) Pub. No.: US 2016/0354992 A1
(43) Pub. Date: Dec. 8, 2016

(54) Therapeutic Or Diagnostic Medical Product Having An Adhesion-Enhancing Surface Structure

(71) Applicant: BIOTRONIK SE & Co. KG, Berlin (DE)

(72) Inventors: Gernot Kolberg, Berlin (DE); Michael Friedrich, Kleinmachnow (DE)

(21) Appl. No.: 15/168,732

(22) Filed: May 31, 2016

(30) Foreign Application Priority Data
Jun. 2, 2015 (DE) 10 2015 108 670.0
Jun. 2, 2015 (DE) 10 2015 108 671.9
Jun. 2, 2015 (DE) 10 2015 108 672.7

(51) Int. Cl. B32B 3/26; A61L 24/00; A61L 24/06; A61L 27/52; A61L 27/18; A61L 27/16; A61L 27/26; A61L 29/14; A61L 29/04; A61L 29/06; A61L 31/04; A61L 31/06; A61L 31/14; B32B 3/06; B32B 3/08; B32B 27/08; B32B 27/32; B32B 27/28; A61L 24/04

(52) U.S. Cl. CPC B32B 3/263; A61L 24/046; A61L 24/0031; A61L 24/06; A61L 24/043; A61L 27/52; A61L 27/18; A61L 27/16; A61L 27/26; A61L 29/145; A61L 29/049; A61L 29/041; A61L 29/06; A61L 31/041; A61L 31/048; A61L 31/06; A61L 31/145; B32B 3/06; B32B 3/08; B32B 27/08; B32B 27/32; B32B 27/283; A61L 2400/18; B32B 2307/7242; B32B 2535/00

(57) ABSTRACT

A therapeutic or diagnostic medical product having an adhesion-enhancing surface structure, wherein the adhesion-enhancing surface structure is part of a membrane covering a cavity filled with a plastically deformable material.

### CROSS-REFERENCE TO RELATED APPLICATIONS

This patent application claims the priority of co-pending German Patent Application Nos. DE 10 2015 108 670.0; DE 10 2015 108 672.7; and DE 10 2015 108 671.9, all filed on Jun. 2, 2015 in the German Patent Office, the disclosures of which are hereby incorporated by reference in their entireties.

### TECHNICAL FIELD

The present invention relates to a therapeutic or diagnostic medical product that has an adhesion-enhancing structure at least on parts of its outwardly facing surface.

### BACKGROUND

The temporary to permanent fixing of therapeutic or diagnostic medical products in humans or animals is of great importance. A fixing of this type is intended, in particular, to prevent a dislodgement of the medical products in or on the body, as a result of which the desired diagnostic or therapeutic function can no longer be ensured, or additional medical complications can even occur. The fixing mechanism itself should have a minimal effect on the organism. In the case of purely mechanical fixing by sewing, or by anchoring structures or clamping elements, the affected tissue may be damaged in a lasting manner and potentially irreparably. Adhesion-enhancing glues can lead to incompatibility reactions, and medical products fixed using such glues generally can no longer be separated from the adhering tissue without damage.

Another reason to fix components in the vascular system or in or on the heart is to hold the components in a secure position. Otherwise, the components would be swept along by the blood flow or, as a result of gravity, would reach locations where they might be dangerous for the patient. They might thus block vessels, resulting in embolisms, heart attack or stroke.

There is thus a need for alternative fixing mechanisms for therapeutic or diagnostic medical products, which fixing mechanisms on the one hand ensure a reliable and trouble-free fixing, but on the other hand can be detached without leaving anything behind.

The present invention is directed toward overcoming one or more of the above-mentioned problems.

### SUMMARY

The discussed problems of the prior art can be solved or at least mitigated with the aid of the therapeutic or diagnostic medical product according to the present invention. The medical product for this purpose has an adhesion-enhancing surface structure—preferably a gecko structure—which is part of an elastic membrane covering a cavity filled with a plastically deformable material.

The present invention thus utilizes an alternative possibility for the connection of different surfaces via the phenomenon of dry adhesivity. Dry adhesivity is understood in the present case to mean the formation of adhesive forces between surfaces without adhesion-enhancing substances, such as, for example, glues. Adhesion systems of this type are also known, for example, from nature, for example in the case of gecko legs or insect legs. It is assumed that in such systems the adhesive forces are based on van-der-Waals forces. The adhesion-generating surface for this purpose has an adhesion-enhancing surface structure, for example, a multiplicity of brush-like or hair-like elements, which lead to a very large increase in the available contact area. With the enlargement of the contact area, the strength of the adhesion forces formed in the event of contact consequently also increases. The use of adhesion-enhancing surface structures of this type for attachment to tissue is proposed for example by Alborz Mandavi et al., ‘A Biodegradable and Biocompatible Gecko-inspired Tissue Adhesive’, PNAS (2008), Vol. 105, No. 7, 2307-2312.

In the case of dry adhesivity, the strength of the adhesion between two surfaces is therefore related to the area available for the adhesion. Two planar surfaces adhere much better to one another than a planar surface and a rough or non-planar surface. Generally, the greater is the area available for adhesion, the better two surfaces will adhere to one another. However, this area could not be altered before now and, therefore, the adhesion force could not be altered. Since the adhesion-enhancing surface structure according to the present invention is part of a membrane that loosely covers the cavity filled with the plastically deformable material, the area available for adhesion can be significantly increased. In other words, the membrane and the plastically deformable material form a sort of gel cushion, such that a surface contour of the membrane can adapt to the surface contour of the tissue.

The adhesion-enhancing surface structure may have between 10 and 1,000,000 rods per square millimeter, for example. The ratio of diameter and length of the rods may be between 1:2 and 1:2,000. The cross section of the rod may be cross-profiled, for example, completely or partially round, triangular, rectangular, square or internally hollow. It may have a T-profile or may correspond to a crescent-shaped outline. A preferred bending direction of the rod can thus be predefined. Alternatively, or in combination, the rods can be pre-bent or obliquely attached. A uniform bending direction of the rods may prevent the rods from becoming entangled with one another. The rods may also have a longitudinal profile. They may thus be thickened at the root, where they bear against the component to be fixed, and may taper toward the end.

The adhesion-enhancing surface structure can consist of rods that branch out. The end of the last branch can be thickened again. The greatest extent of the thickened portion corresponds at most to 100 times the rod diameter on which the thickened portion sits. The end of the last branch may also be planar or rounded or pointed. A lobe-like structure, similarly to a scoop, can be located at the end of the last branch and is attached at one end. The lobe-like structure is preferably attached at one end to the rods in such a way that the angle of the rods is continued. In the event of a transverse force of the component in the detaching direction (for example, in an anticlockwise direction), the lobe-like structure peels away from the tissue, which significantly facilitates the detachment, whereas in the event of transverse force in the other direction only a shear force is caused, which not only does not detach the fixing, but aids the fixing.

The fixing and detachment forces can be set by organization of the bending direction of the rods on the surface. The structures are fixed particularly well when as many rods as possible absorb the tensile forces simultaneously. If the fixing is to be released, the rods must be individually loaded, where possible, so as to enable a detachment even with low forces. Due to the preferred bending direction of the rods, a force acting laterally on the component can be converted into a tensile force or into a compressive force, depending on direction. A force against the rod orientation leads to a force compressing the rod, which causes the rod to bend, as a result of which a rolling motion occurs at the fixing surface, which peels off the fixing surface. This effect can also be utilized over a number of rod sections. For example, only the lower end of the rods may thus be provided with a preferred direction. The subsequent, for example, branched structures are peeled off. An equivalent effect is attained when the rods do not have a preferred bending direction, but are already obliquely attached or pre-curved.

A special embodiment of the rods, which are pre-bent or provided with a preferred bending direction, is one in which the rods are pre-bent about a pivot point, preferably the point of the electrically active or sensitive area in one direction, preferably in an anticlockwise direction. A rotation at the component in an anticlockwise direction rolls each individual rod end about the fixing point and peels it off. The fixing can thus be provided by pressing the component on, or by rotation in a clockwise direction. Detachment occurs by rotation in an anticlockwise direction.

Besides the specified tangential orientation of the rods, further structured arrangements are conceivable, for example, an area in which the rods point in one direction is detachable by a force in this direction and is stable in the other direction.

If the component is to be detached by means of an orthogonally acting force, it is expedient for the rods to point towards the membrane center point. If the component is removed perpendicularly, the rods detach from the outside in. This process can be triggered alternatively by a ram, which presses from the inside onto the membrane, or by fluid pressure.

The adhesion-enhancing surface structure can be manufactured in principle from any material that can be connected to the further constituents of the membrane and that is sufficiently compatible for an intracorporeal use. The adhesion-enhancing surface structures preferably consist of a polymer material, in particular, a silicone. Further possible materials for the structures include, for example, carbon materials, in particular, in the form of fibers and nanotubes, polypropylene, polytetrafluoroethylene (PTFE), ethylene tetrafluoroethylene (ETFE), polycarbonate, polystyrene, polylactides, for example PDLLA, synthetic spider silk, polyurethanes and copolymers thereof, polyimide, polyamide, polyether ether ketone (PEEK), polysulfone, polyethylene, polyoxymethylene (POM), polyether block amide, chitin, collagen, cellulose, keratin, metals, glass, and ceramic. The adhesion-enhancing surface structures may consist, in particular, of an electrically conductive material so as to also enable electrical contact in addition to the mechanically stable contact. The structure can be coated by a suitable substance, such as, for example, poly(dopamine methacrylate-co-2-methoxyethyl acrylate) (p(DMA-co-MEA)), so as to improve the adhesive strength in liquid media, or with steroids, so as to suppress inflammation processes. Substances that promote ingrowth behavior can also be used.

The membrane can be constructed in a multi-layered manner, wherein the outermost layer has the adhesion-enhancing surface structure. The one or more inner layers of the membrane are then preferably formed from a polymer material, in particular, a polyethylene. The one or more inner layers of the membrane can be optimized in this way in respect of the properties desired for the respective application, irrespectively of the adhesion-enhancing surface structure. The membrane, however, may optionally also consist of the same material from which the adhesion-enhancing surface structure is formed. In this case a multi-layered membrane structure can be omitted.

An adhesion-enhancing surface structure can be produced by different methods. By way of example, negative molds can be produced by lithographic methods, such as electron beam lithography and laser lithography, or by etching methods. In a subsequent casting method the positive surface with hair-like extensions is then produced starting from the negative mold (for example, see A.K. Geim et al., Nature Mater. 2, 461-463 (2003) and H. Lee, B.P. Lee and P.B. Messersmith, Nature 448, 338-341 (2007)).

The medical product is preferably an implantable sensor, a heart electrode, a heart valve, a medication pump, a catheter (for example, an electrophysiological catheter, ultrasound catheter, balloon catheter, ablation catheter, urinary catheter or RSD catheter), a stent, a gastric tube, a vascular repair aid (what are known as patches), an external skin electrode (for example, electrodes for ECG or muscle stimulation), a sticking plaster, an orthodontic brace, or a prosthesis.

Further embodiments, features, aspects, objects, advantages, and possible applications of the present invention could be learned from the following description, in combination with the Figures, and the appended claims.

Further preferred embodiments of the present invention will emerge from the dependent claims and the following description.

### DESCRIPTION OF THE DRAWINGS

The present invention will be described hereinafter on the basis of an exemplary embodiment and associated drawings, in which:

FIG. 1 shows a schematic illustration of a medical product according to the present invention in the form of an implantable sensor.

FIG. 2 shows a sectional view through a region of the sensor from FIG. 1, which has a membrane having an adhesion-enhancing surface structure.

FIG. 3 shows a schematic sectional view through a further embodiment of the membrane with adhesion-enhancing surface structure.

### DETAILED DESCRIPTION

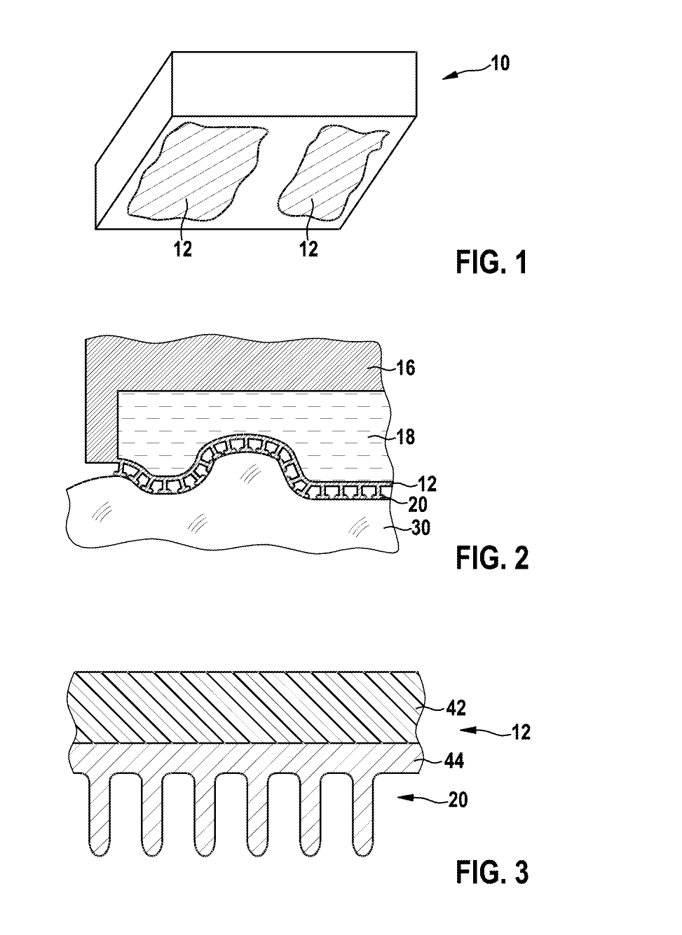

FIG. 1 heavily and schematically illustrates an implantable sensor 10, which is provided with the means according to the present invention for intracorporeal fixing. The sensor 10 can be designed, for example, in such a way that it can detect electrophysiological processes in an adjacent tissue via electrodes and can transmit data to an external reader by means of wireless transmission technology (not illustrated here in greater detail).

The fixing means according to the present invention are arranged here by way of example in two regions of a longitudinal side of the sensor 10 and each comprise a membrane 12 having an adhesion-enhancing surface structure explained hereinafter in greater detail. The irregular edge contour of the elastic membrane 12 that can be inferred from the schematic illustration is intended to show that the membrane 12 does not bear against the sensor 10 in a tautly tensioned manner, but is deformable in terms of its surface contour.

FIG. 2 shows a detail through the sensor 10 of FIG. 1. A housing part 16 of the sensor 10 carrying the fixing means according to the present invention here has a cavity 18. This is hermetically covered by the membrane 12, wherein the membrane 12 is not fixedly braced, however, over the edges of the cavity 18, but still has a certain amount of play. The interior of the cavity 18 is filled with a plastically deformable gel. The membrane 12 and the filled cavity 18 thus form a gel cushion. The adhesion-enhancing surface structures 20, which are intended to enable an attachment to the tissue 30, are disposed on the outer side of the membrane 12. The adhesion-enhancing surface structures 20 preferably cover the entire outer surface of the membrane 12.

Here, the adhesion-enhancing surface structures 20 are provided purely by way of example in the form of rods having a head that is widened in a plate-like manner. The specific embodiment of the adhesion-enhancing surface structure 20 may vary, however, for example, depending on the intended application. In particular, a gecko structure recreating nature is provided on the outer side of the membrane 12. The adhesion-enhancing surface structure 20 can be produced, as already mentioned further above, in a manner known per se by creating matching negative molds for a plastics casting method.

As can be seen from the schematic illustration of FIG. 2, the membrane 12 adapts to the surface contour of the tissue 30 as the sensor 10 is pressed there against. The contact area between the two surfaces necessary for the adhesion is significantly increased as a result.

FIG. 3 shows a further embodiment of the membrane 12. The membrane 12, which is illustrated in section, is constructed in two layers. A first layer 42 consists of a polyethylene film approximately 0.02 to 0.1 mm thick. The first layer 42 is coated by a second layer 44, which has the adhesion-enhancing surface structure 20. The second layer 44 consists of silicone and is 0.02 to 0.2 mm thick. Here, the adhesion-enhancing surface structure 20 has a multiplicity of rod-shaped extensions.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. A therapeutic or diagnostic medical product comprising an adhesion-enhancing surface structure, wherein the adhesion-enhancing surface structure is part of a membrane covering a cavity filled with a plastically deformable material.

2. The medical product according to claim 1, wherein the plastically deformable material is a gel.

3. The medical product according to claim 1, wherein the adhesion-enhancing surface structure is formed from a polymer material.

4. The medical product according to claim 3, wherein the polymer material is a silicone.

5. The medical product according to claim 1, wherein the membrane is constructed in a multi-layered manner and the outermost layer has the adhesion-enhancing surface structure.

6. The medical product according to claim 5, wherein one or more inner layers of the membrane are formed from a polymer material.

7. The medical product according to claim 6, wherein the polymer material is a polyethylene.

8. The medical product according to claim 1, wherein the adhesion-enhancing surface structure is a gecko structure.

9. The medical product according to claim 1, wherein the medical product is an implantable sensor, a heart electrode, a heart valve, a medication pump, a catheter, a stent, a gastric tube, a vascular repair aid, an external skin electrode, a sticking plaster, an orthodontic brace, or a prosthesis.
